(19) United States
(12) Patent Application Publication
(10) Pub. No.: US 2012/0271352 A1
(43) Pub. Date: Oct. 25, 2012

(54) FACET JOINT IMPLANT

(75) Inventors: Martin Schulze, Munster (DE); Ulrike Kettenberger, Trostberg (DE)

(73) Assignee: Aesculap AG, Tuttlingen (DE)

(21) Appl. No.: 13/432,746

(22) Filed: Mar. 28, 2012

(30) Foreign Application Priority Data
Mar. 28, 2011 (DE) 10 2011 001 590

(51) Int. Cl. A61B 17/70

(52) U.S. Cl. 606/247

(57) ABSTRACT

A facet joint implant includes a joint surface replacement implant and a fastening device for fixing the joint surface replacement implant in the joint gap of a facet joint of a human or animal spinal column. The fastening device includes at least two fastening elements that protrude from the joint surface replacement implant. The at least two fastening elements are each provided in the region of their free ends with at least one fixing element through hole. The facet joint implant includes a fixing element which is fed through at least two fixing element through holes of at least two fastening elements. The free ends of the fixing element are connectable to one another.

### CROSS-REFERENCE TO RELATED APPLICATIONS

The present disclosure relates to the subject matter disclosed in German application number 10 2011 001 590.6 of Mar. 28, 2011, which is incorporated herein by reference in its entirety and for all purposes.

### FIELD OF THE INVENTION

The present invention relates to facet joint implants generally, and more specifically to a facet joint implant comprising a joint surface replacement implant and a fastening device for fixing the joint surface replacement implant in the joint gap of a facet joint of a human or animal spinal column, which fastening device comprises at least two fastening elements which protrude from the joint surface replacement implant.

### BACKGROUND OF THE INVENTION

Facet joints can be a significant reason for the onset of back pain. The replacement of facet joints due to the degeneration thereof or damage thereto is therefore an important topic in orthopaedics and especially in the field of spinal column surgery. In many cases, facet joint arthrosis or degeneration is a natural consequence of the reduction in height of a degenerated vertebral disk that is associated with the facet joint concerned i.e. the neighbouring one in particular, whereby there is a change in the load distribution on a vertebral motor segment, also called vertebral motion segment and formed between two neighbouring vertebrae of the spinal column. However, the change in the facet joint could also be representative of an iatrogenic illness resulting from a spinal decompression or the use of a vertebral disk prosthesis.

For the purposes of treating facet joint arthrosis or degeneration, one can effect replacement of the entire facet joint or at least replacement of the joint surfaces after removing the pain-inducing joint capsule and the cartilage layer. Since the facet joints in the vertebral motor segments of the spinal column have an important function to fulfill in regard to load transfer and limitation of the extent of movement particularly of axial rotation and flexion, preservation of the natural extent of movement (“range of motion”) and of the natural distribution of load in the vertebral motor segment appears to be important for the success of such an implant.

The fixation of every implant of this type, however, represents a special challenge since it is the primary stability in particular which substantially determines the functionality of the implant after the implantation thereof.

Facet joint implants of the type described above are known from WO 2009/006455 A1 and also US 2005/0055096 A1 for example. In particular, these facet joint implants are fixed to the bone by means of screws or other fastening elements. Such a method of fixing the facet joint implant is, however, time-consuming and involves risks. In particular, the possibility of damage to the neighbouring bone substance is always present in the case of the aforementioned fastening elements.

Therefore, it would be desirable to provide a facet joint implant which is affixable to the spinal column in a simple and secure manner.

### SUMMARY OF THE INVENTION

In accordance with the invention a facet joint implant comprises a joint surface replacement implant and a fastening device for fixing the joint surface replacement implant in the joint gap of a facet joint of a human or animal spinal column. Said fastening device comprises at least two fastening elements that protrude from the joint surface replacement implant. The at least two fastening elements are each provided in the region of their free ends with at least one fixing element through hole. The facet joint implant comprises a fixing element which is fed through at least two fixing element through holes of at least two fastening elements, wherein the free ends of said fixing element are connectable to one another.

### BRIEF DESCRIPTION OF THE DRAWING FIGURES

The foregoing summary and the following description may be better understood in conjunction with the drawing figures, of which:

FIG. 1: shows a schematic view of a vertebral motor segment of a human spinal column with a facet joint implant;

FIG. 2: an enlarged view of the region A in FIG. 1;

FIG. 3: a perspective view of the facet joint implant illustrated in FIGS. 1 and 2 in a basic position;

FIG. 4: a schematic view of a further exemplary embodiment of a fastening element for a facet joint implant;

FIG. 5: a schematic view of a further exemplary embodiment of a fastening element for a facet joint implant;

FIG. 6: a schematic view of a further exemplary embodiment of a fixing element locking arrangement for a facet joint implant;

FIG. 7: a partially schematic sectional view through a joint surface replacement implant of a facet joint implant;

FIG. 8: a schematic view of a further exemplary embodiment of a fastening element for a facet joint implant;

FIG. 9: a schematic view of a further exemplary embodiment of a fastening element for a facet joint implant;

FIG. 10: a schematic view of a further exemplary embodiment of a joint surface replacement implant for a facet joint implant;

FIG. 11: a schematic view of a film hinge formed between a fastening element and a joint surface replacement implant of a facet joint implant; and

FIG. 12: a schematic view of a hinge joint formed between a fastening element and a joint surface replacement implant of a facet joint implant.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a facet joint implant comprising a joint surface replacement implant and a fastening device for fixing the joint surface replacement implant in the joint gap of a facet joint of a human or animal spinal column, which fastening device comprises at least two fastening elements that protrude from the joint surface replacement implant, wherein the at least two fastening elements are each provided in the region of their free ends with at least one fixing element through hole, and in that the facet joint implant comprises a fixing element which is fed through at least two fixing element through holes of at least two fastening elements, wherein the free ends of said fixing element are connectable to one another.

A facet joint implant in accordance with the invention of this type makes it possible to achieve optimal matching to the bony structures in the region of the facet joint that is being treated. The means for affixing the facet joint implant is simple since it is possible, in particular, to form it from just two parts. For example, the fixing element can be fed through the fixing element through holes of all the fastening elements and then be connected to itself in a simple manner. This permits rapid, simple and secure attachment of the facet joint implant to the existing bone without causing damage thereto. There is no compelling need for additional fastening elements which are driven into the bone, although these could also be provided if so required. Furthermore, it is also possible with the aid of this facet joint implant in particular not to endanger the neural structures which frequently border onto facet joints. Moreover, the proposed fastening device in particular can also prevent the facet joint implant from slipping off the bony structures. In principle, it is also conceivable for the facet joint implant formed in accordance with the invention to be inserted into the body of the patient through a minimally invasive point of entry. Due to this simple form of fixation of the facet joint implant, combinations thereof with other measures for stabilizing the spinal column particularly in the region of the vertebral motor segment concerned are possible without giving rise to problems in practice. In addition, as there is no compelling need for bony structures to be damaged or destroyed when fixing the facet joint implant in accordance with the invention, it is still possible for neighbouring vertebrae to be subsequently fused together after the insertion of the proposed facet joint implant. And finally, the proposed facet joint implant also makes it possible in particular to take into consideration the physiological state of a patient in order to achieve as gentle an affixation of the facet joint implant as possible.

It is expedient, if the fixing element is flexible. It is thereby possible, in particular, for the facet joint implant to be adapted and fixed to almost any conceivable joint surface. Furthermore, handling of the facet joint implant is also very simple.

Preferably, the fixing element is in the form of a thread or a wire. Such a fixing element permits it to be easily handled and also permits affixation of the facet joint implant or latching thereof to bony structures of the spinal columns from the dorsal side.

It is particularly advantageous if the at least two fastening elements are in the form of fastening straps, tabs, strips, loops or eyelets. The production of fastening elements of this type is particularly simple. Furthermore, they enable further optionally required fixing element through holes to be provided in a simple manner and for them to be formed in such a manner that the fixing element can be passed securely therethrough.

It is expedient if free ends of the at least two fastening elements are thickened and/or reinforced or if the at least two fastening elements are thickened and/or reinforced in the region of their free ends. Fastening elements formed in such a manner can ensure, in particular, long term stability for the employment of the facet joint implant. Rupturing of the fastening elements due to tensile stress applied by the fixing element can thereby be prevented in a simple and secure manner.

In particular, rupturing of the facet joint implant in the region of the fixing element through hole can be prevented in an advantageous manner by thickening and/or strengthening the fixing element through hole.

In accordance with a further preferred embodiment of the invention, a fixing element locking arrangement can be provided for connecting the free ends of the fixing element by the application of a tensile-load. With the aid of the fixing element locking arrangement, it is possible in a simple manner to connect the free ends of the fixing element together under tension in order to fix the facet joint implant to the existing bony structures of the spinal column.

The facet joint implant can be fixed to the spinal column in a particularly simple, rapid and secure manner, if the fixing element locking arrangement is in the form of a knot, a seal or a clip.

Preferably, the facet joint implant comprises four to 25 fastening elements. The more fastening elements there are provided, the more optimal is the fixture of the facet joint implant to existing bony structures. In particular, fitting the fastening elements very close to the bone prevents impairment of the surrounding structures, such as nerve roots for example.

Moreover, it can be advantageous if the at least two fastening elements are of differing length and/or breadth and/or thickness. The facet joint implant can then be adapted still more optimally to the bone structures of the spinal column. It can also be particularly expedient if the at least two fastening elements are formed such as to be irreversibly separable from the joint surface replacement implant. For example, an operating surgeon can then prepare the facet joint implant for a patient on an individual basis whilst still in the process of the surgical procedure by, for example, separating the unnecessary or impeding fastening elements from the joint surface replacement implant, by tearing or cutting them off for example.

A further, improved, individually applicable adaptation of the facet joint implant to the physiological state of a patient can be achieved, in particular, in that at least two fastening elements are in the form of different kinds of fastening elements.

For example, one fastening element can be in the form of a fastening strap, another in the form of a fastening tab, another in the form of a fastening strip, another in the form of a fastening loop or another in the form of a fastening eyelet. Depending upon the desired position of the facet joint implant on the bone, different kinds of fastening elements can be more expedient, this also applying for example to the guidance of the fixing element.

In order to prevent insofar as possible the facet joint implant from slipping whilst fixing it with the aid of the fixing element, it can be expedient if the facet joint implant comprises at least one retaining element for digging into bone tissue. In particular two, three or a plurality of retaining elements can be provided.

Maintenance of the desired position of the facet joint implant can be further improved, in particular, by arranging or forming at least one retaining element on the joint surface replacement implant and/or on at least one of the fastening elements. Slipping down or slippage of the facet joint implant on the spinal column can thereby be prevented in a simple and secure manner. In particular, two, three or more retaining elements can be provided on the joint surface replacement implant and/or on at least one of the fastening elements.

The construction and also the manufacture of the facet joint implant is particularly simple, if the at least one support element is in the form of a barb or a pointed projection. For example, the projection can be needle-shaped or pyramid-shaped.

In order for the facet joint implant to remain in the body over the long term, it is expedient if it is made of a body-compatible material. Preferably, the joint surface replacement implant and/or the fastening device are made entirely or partly of a metal or a synthetic material. For example, it can be advantageous to make the joint surface replacement implant of a synthetic material and the fastening device of a metal or vice versa.

Advantageously, the metal is or contains titanium or a titanium alloy. Metals of this type exhibit the mechanical sturdiness that is necessary for the construction of the facet joint implant. In addition for example, the joint surface replacement implant can be in the form of a metal foam or be formed with a porous surface which improves the growth and integration of the bone.

The production of the facet joint implant can be further simplified, in particular, if the synthetic material is or contains polyetheretherketone and/or polyethylene. The aforementioned synthetic materials are particularly suitable for use as implant materials and they also exhibit the mechanical sturdiness required for the construction of a facet joint implant. Preferred in particular is UHMWPE i.e. polyethylene having an ultrahigh molecular weight.

Furthermore, it can be expedient if the synthetic material is a thermally activatable and/or a thermally shrinkable synthetic material. In particular, this permits the facet joint implant to be positioned in the desired manner and then shrinkage to be effected by the provision of heat, for example of the fastening elements. This can be used for tensioning the fastening elements and the joint surface replacement implant relative to each other and thereby lead to still better retention of the facet joint implant on the spinal column.

It is advantageous, if parts or regions of the joint surface replacement implant and/or the fastening device that are made from a synthetic material are provided at least partly with an osteo-integrative coating. Preferably, the osteo-integrative coating is provided on the joint surface replacement implant and/or on the fastening device at those positions or areas which can come into contact with the bone during the implantation process. The osteo-integrative coating serves to improve the growth and integration of bone into the joint surface replacement implant and/or the fastening device. Preferably, it can be porous and made of a metal or contain a metal or a metal alloy. For example, the coating may contain titanium. Expediently, the coating is produced by means of a cold gas injection process.

The manipulability of the facet joint implant and also the production process therefore can be further improved in a simple manner if the at least two fastening elements are flat or slightly curved in a basic position.

In order to introduce the facet joint implant into the body of a patient through a minimally invasive point of entry in a simple manner and to individually adapt it in a desired manner to the physiological state of the patient, it is advantageous if the at least two fastening elements are flexible and/or resilient. In this sense, flexible implies, in particular, a deformability to the extent that it is possible to bend or twist the fastening elements for example.

Furthermore, in accordance with a further preferred embodiment of the invention, provision may be made for the at least two fastening elements and the joint surface replacement implant to be connected to one another in articulated manner. Pulling and retaining forces can thereby be initiated in an optimised manner in dependence on the design of the facet joint implant and also as dependent on the physiology of the patient.

It is advantageous, if the facet joint implant comprises a joint for the articulated connection of one of the fastening elements to the joint surface replacement implant. Such an arrangement is particularly appropriate if it is desirable to have different combinations of material between the joint surface replacement implant and the fastening element, for example, in order to form a suitable sliding fit of the replacement joint surfaces defined by the cooperating joint surface replacement implants whilst permitting flexible fixing at the same time.

It is particularly expedient, if the joint is in the form of a hinge joint. A one-piece hinge joint can preferably be in the form of a film hinge.

The production and also the stability of the facet joint implant can be improved in particular if, as a whole, it is formed in one piece manner. In particular hereby, one piece manner means that the joint surface replacement implant and the at least two fastening elements are formed in one piece. However, the fixing element can be provided as a separate part.

In accordance with a further preferred embodiment of the invention, provision may be made for the joint surface replacement implant and the at least two fastening elements to be formed in one piece manner. Thus, for example, the joint surface replacement implant can be formed together with the fastening elements in a single manufacturing step.

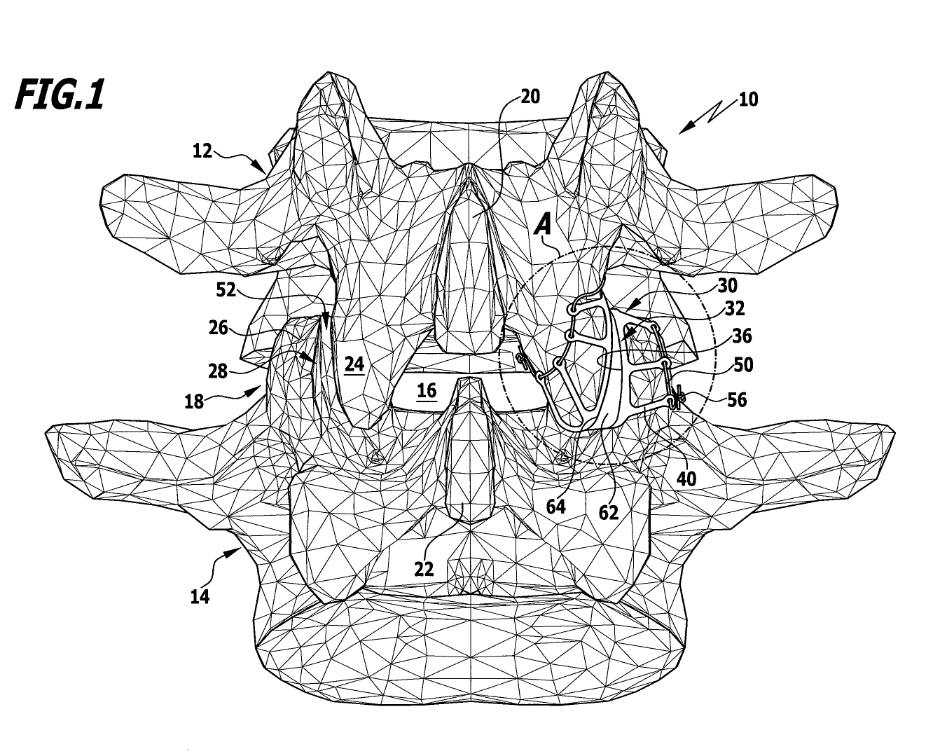

A part of a human spinal column 10 is illustrated schematically in FIG. 1. The two neighbouring vertebrae 12 and 14 together with the vertebral disk 16 arranged therebetween form a vertebral motor segment of the spinal column 10 which bears the general reference symbol 18. Located laterally of and adjacent the spinous processes 20 and 22 of the vertebrae 12 and 14 in the dorsal direction are the mutually facing processes 24 and 26 which, together, form a respective facet joint 28. Thus each vertebral motor segment 18 comprises two facet joints 28 that are symmetrical to the median plane.

The facet joint 28 illustrated to the right in FIG. 1 was partly replaced by one or two facet joint implants 30 for the purposes of treating facet joint arthrosis or degeneration. Each facet joint implant 30 comprises a joint surface replacement implant 32 which comprises a contact surface 34 located on a respective one of the processes 24 and 26 and also a joint surface 36 pointing in the opposite direction, i.e. in the direction of the other respective process 24, 26. If, as is schematically illustrated in FIG. 1 to the right in the region A, a facet joint implant 30 is attached to each process 24 and 26, then the respective joint surfaces 36 of the joint surface replacement implants 32 face one another.

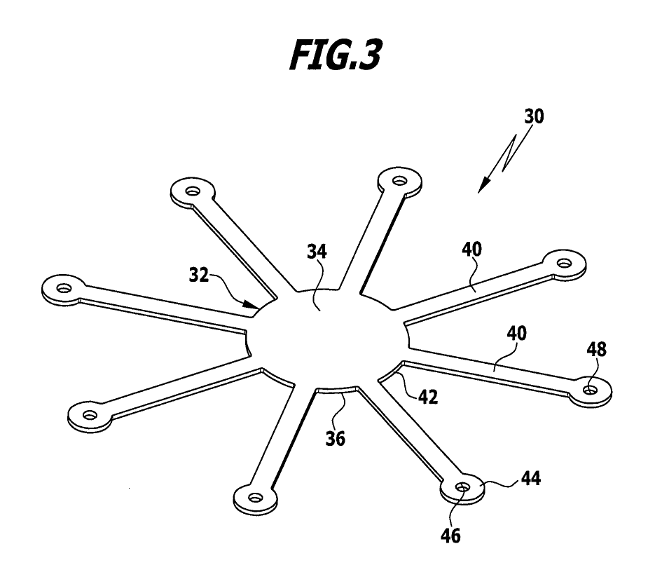

For the purposes of affixing the facet joint implant to the process 24, 26, there serves a fastening device 38, 30. This comprises at least two, preferably four to 25 fastening elements 40 which protrude from an edge 42 of the joint surface replacement implant 32 which is substantially in the form of a circular disk. The facet joint implant 30 as a whole is formed in one piece manner and, in a basic position that is illustrated in FIG. 3, particularly immediately after it has been produced, it is substantially flat.

The outer diameters of the free ends 44 of the fastening elements 40 are somewhat thickened and incorporate a through hole 46 in the form of a boring. This forms a fixing element through hole 48.

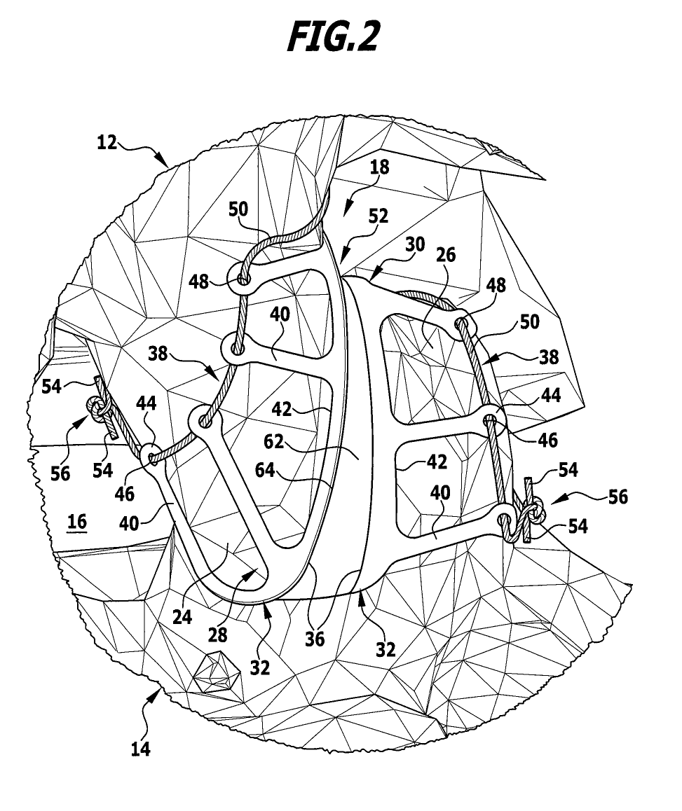

As schematically illustrated in FIG. 2 and as is readily apparent therein, there is provided a fixing element 50 in the form of a flexible thread or wire which is fed through all the fixing element through holes 48.

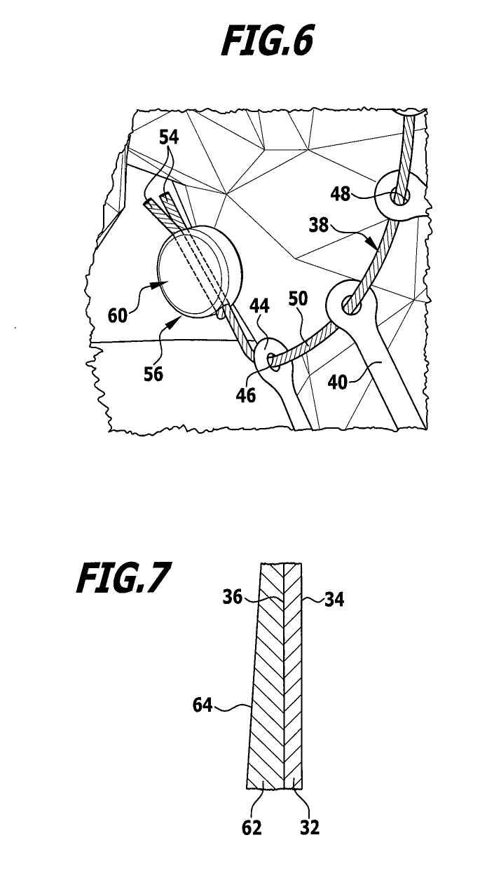

For the purposes of affixing the facet joint implant 30, the contact surface 34 of the respective joint surface replacement implant 32 is advanced onto the damaged or degenerated joint surface of one of the processes 24 and 26 in the joint gap 52 of the facet joint 28. The damaged joint surface can be additionally prepared beforehand. The fastening elements 40 are bent away from the respective other process 24, 26 and the fixing element 50 that has been fed through the fixing element through holes 48 is tightened around the respective process 24, 26. The free ends 54 of the fixing element 50 are connected together. This can be done by means of a fixing element locking arrangement 56 which is producible by the application of a tensile load and may be in the form of a knot for example, as is schematically illustrated in FIGS. 1 and 2, or it may be in the form of a seal 60 which, in particular, is arranged to be pushed or crimped on, as is illustrated schematically in FIG. 6. Alternatively, clips for connecting the free ends 54 are also conceivable.

As is schematically illustrated in FIG. 3, the joint surface replacement implant 32 can be in the form of a flat disk. Alternatively, the joint surface 36 of the disk could also carry a spacer element 62 which, in turn, defines a separate joint surface 64 that faces away from the joint surface replacement implant 32 and its joint surface 36. This is schematically illustrated in the form of a partial sectional view in FIG. 7. Thus, for example, the joint surface 64 of the spacer element 62 can abut the joint surface 36 of a corresponding facet joint implant 30. The spacer element can be cylindrical and/or wedge-shaped.

Ends of the fastening elements 40 remote from the joint surface replacement implant can also be in the form of a loop 66, as is schematically illustrated in FIG. 5. Hereby, one free end 68 of the fastening element 40 is turned back on itself and fixed by means of a rivet 70 or a spot weld for example. Again, a fixing element through hole 48 is defined by the loop 66 which is oriented in a direction parallel to a plane defined by the joint surface replacement implant 32.

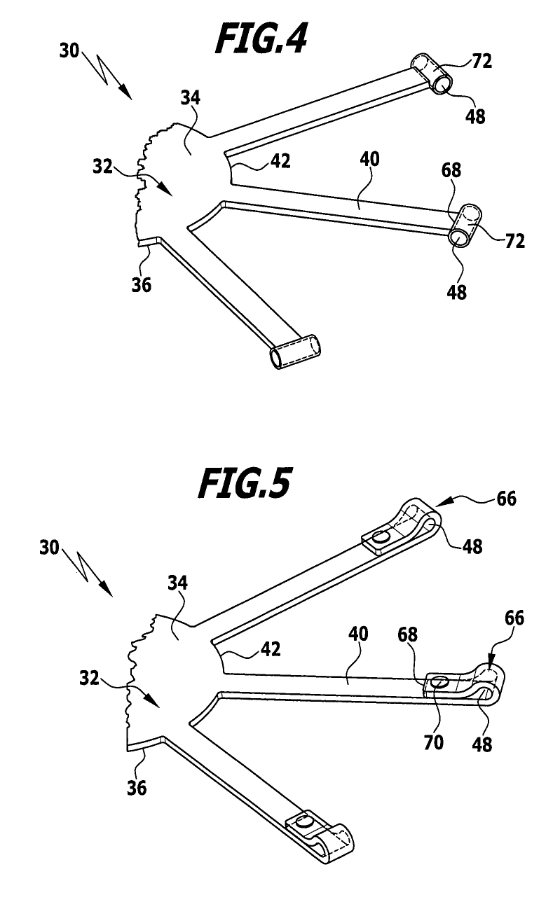

A further variant of a fixing element through hole 48 is illustrated schematically in FIG. 4. A sleeve-like eyelet 72 is formed on or attached to a free end 68 of the strip-like, or strap-like fastening element 40, said eyelet likewise defining a fixing element through hole 48 that is oriented in a direction parallel to the plane defined by the joint surface replacement implant 32.

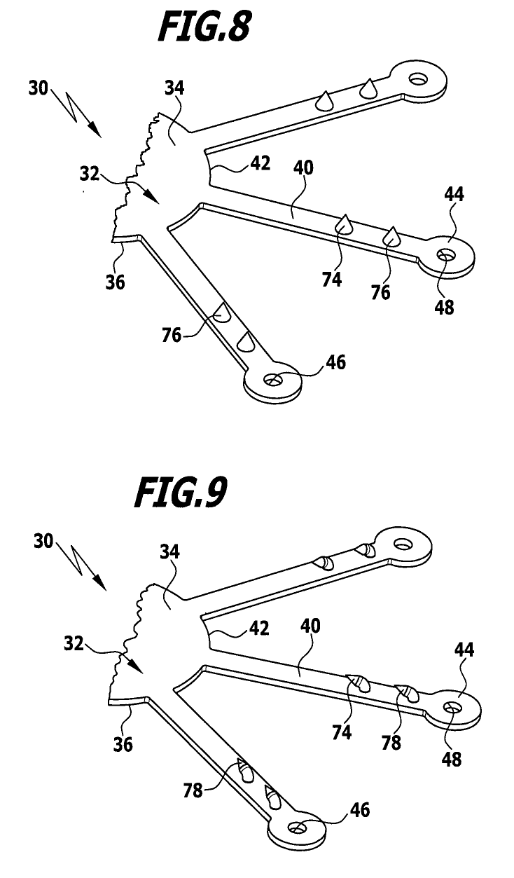

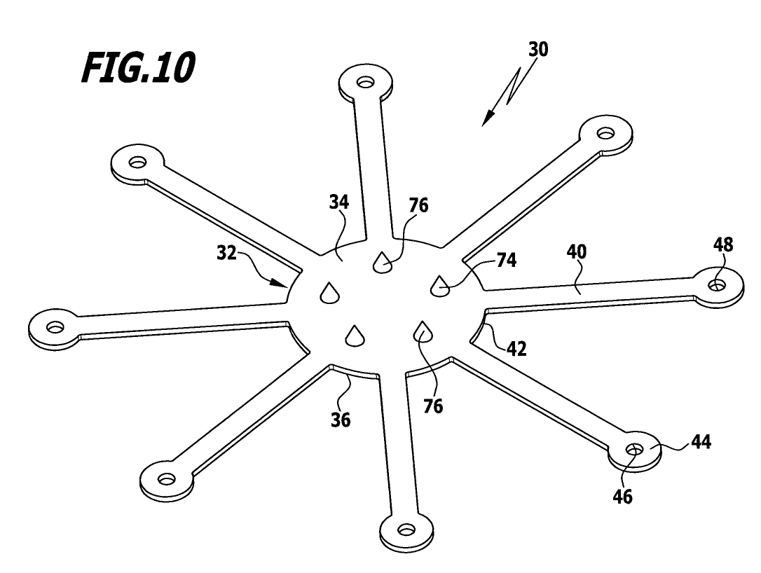

In order to exclude the possibility of slippage of the facet joint implant 30 on the processes 24 or 26 insofar as possible, it is expedient for the facet joint implant 30 to carry one or more retaining elements 74. These can be arranged on the contact surface 34 as is schematically illustrated in FIG. 10 for example, and they are formed such as to protrude perpendicularly or substantially perpendicularly therefrom. Moreover, as is schematically illustrated in FIGS. 8 and 9, the retaining elements 74 could also be in the form of pointed projections 76 or barbs 78 and be arranged or formed on a fastening element 40 such as to protrude therefrom. The projections 76 can, in particular, be in the form of cone tips or pyramid tips. Two, three, four, five or more retaining elements 74 may project from the contact surface 34. Each fastening element 40 can comprise one, two, three, four or more retaining elements 74.

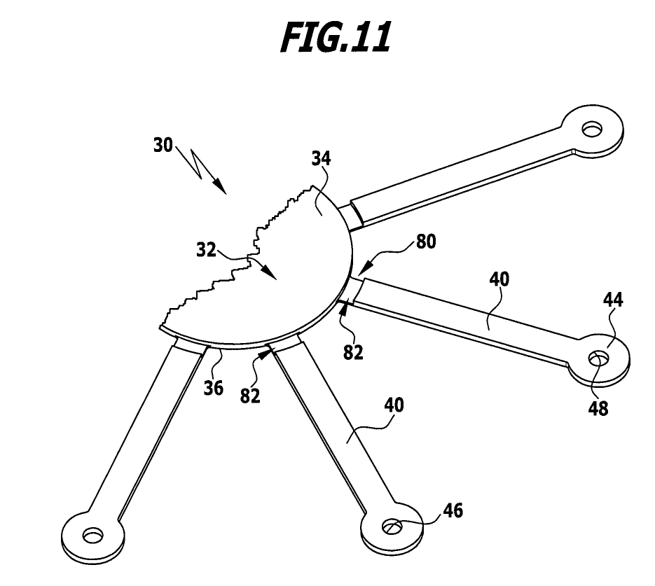

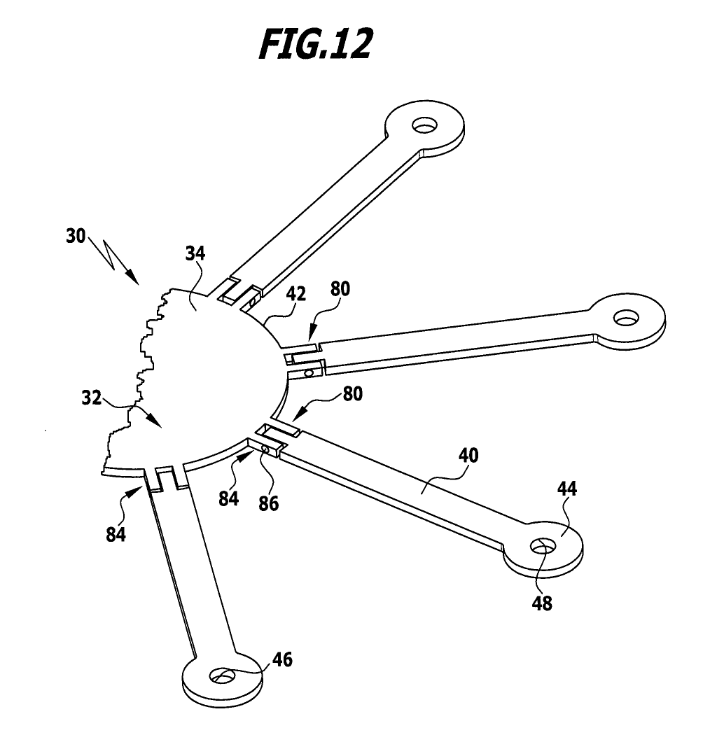

For improving the mobility and thus deformability of the facet joint implant 30 for the purposes of adapting it to the processes 24, 26 in the best possible manner, a joint 80 can be formed between the joint surface replacement implant 32 and the fastening element 40. Hereby, as is illustrated schematically in FIG. 11, this joint may be a film hinge 82 which is formed by means of a weakened area in the region between the fastening element 40 and the joint surface replacement implant 32. Alternatively, the joint 80 could also be in the form of a real hinge joint 84. This can, in particular, comprise a hinge pin 86 which passes through a recess not shown in further detail in the joint surface replacement implant 32 and forms a bearing shaft for a bearing through hole that is provided on the fastening element 40 and corresponds to the hinge pin 86. A pivotal movement of the fastening element 40 about a pivotal axis defined by the hinge pin 86 is thereby possible.

The different embodiments of the fastening elements 40, and in particular too, the different fixing element through holes 48 thereof that have been described above can be combined with one another on a facet joint implant 30 in any desired manner. In addition, the fastening elements can be implemented with differing thicknesses and/or lengths. Furthermore, it is conceivable for the facet joint implant 30 to be constructed in such a way that individual fastening elements 40 can be removed during the implantation process, for example, for them to be cut off or torn off. As an option furthermore, the free ends 44 and 68 of the fastening elements 40 can also be reinforced or thickened in order to prevent them from rupturing as a result of a tensile load applied by means of the fixing element 50.

Furthermore, further options for improving the affixation of the described facet joint implants 30 to the processes 24 and 26 are not illustrated. In order to enable the joint surface replacement implant 32 to be inserted into the joint gap 52, the capsule of the facet joint 28 has to be at least partly removed. The preparation of the facet joint 28 depends in particular on the actual bone structure of the patient, i.e. in some cases, it may be necessary to prepare the bone by providing one or more small notches. These notches assist in preventing subsequent slippage of the fastening device 38, and in particular, of the fixing element 50. They grow together following the operation since the abraded bone is rebuilt by the body. Alternatively, for the purposes of increasing the stability of the inserted facet joint implant 30, it is also conceivable to provide further fastening elements such as bone pins or screws for example via which the fixing element 50 can be additionally guided or which additionally fix the fastening elements 40 to the bone.

Self evidently, provision may also be made for two or more fixing elements 50 per facet joint implant 30 which are only fed through a portion of the fixing element through holes 48 and can be connected to themselves or to a further fixing element 50.

The fastening device 38 or the facet joint implant 30 is producible using any of the usual implant materials. For example, titanium, titanium alloys, polyetheretherketone, polyethylene of ultrahigh molecular weight (UHMWPE) are suitable for this purpose. Moreover, these implant materials can also be additionally provided with suitable coatings in order to enable regrowth of the bone and prevent slippage of the joint surface replacement implant 32 on the processes 24 to 26 insofar as possible. Moreover, it would also be conceivable to use an implant material, and in particular a synthetic material, which is caused to shrink by applying heat thereto after the insertion process. After the facet joint implant 30 had been positioned, one would then cause the fastening elements 40 for example to shrink by the application of heat, this thereby leading to the fastening elements 40 and the joint surface replacement implant 32 being braced relative to each other thus catering for still better retention.

The placement of the facet joint implant 30 as a form of covering over a process 24, 26 can thus be compared, in particular, with a cover or hood over a cylindrical article. The fastening elements 40 are preferably flat or slightly curved for the purposes of such placement.

All of the described facet joint implants 30 can be handled in a simple and secure manner. Due to the simple tensioning mechanism of the fastening device 38, the primary stability of the artificial facet joint can be ensured without causing damage to the surrounding structures. The affixation or the process of fixing the facet joint implant 30 can be adapted to the bony structures of the patient i.e. particularly to the processes 24, 26 in just a few steps. Nevertheless, it may be necessary in some cases to prepare the bone due to the morphologic variety of the bone structure, although this is limited to the small notches in the bone that have already been mentioned.

The proposed facet joint implants 30 have an extremely flexible shape and make it possible to adapt them to almost any conceivable joint surface. The insertion of the facet joint implants 30 into the body can be effected using a minimally invasive procedure this thereby making it easier for the patient. In particular, the implantation process described above enables maximum retention of the bony structure so that vertebral body structures, such as the pedicle of the vertebrae for example, which are important for corrective procedures do not have to be damaged. All of the constructions described are economical to produce, simple and functionally secure. They are easy to handle and permit an interlocking process, i.e. affixation to be effected, particularly too, from dorsal.

In particular, independently of the form or the concrete arrangement, all of the facet joint implants 30 described above can be provided partially or entirely with an osteo-integrative coating 88 especially if they are made entirely or partly of a synthetic material. Preferably, the osteo-integrative coating 88 is provided on outer surfaces of the joint surface replacement implant 32 and/or of the fastening device 38 at those positions or areas which can come into contact with the bone during the implantation process. This is illustrated schematically in FIG. 3. The osteo-integrative coating 88 enables improved growth and integration of the bone into the joint surface replacement implant and/or the fastening device. It can preferably be porous and made of a metal and or contain a metal or a metal alloy. For example, the coating can contain titanium. Expediently, the coating 88 is produced by means of a cold gas injection process.

1. A facet joint implant comprising a joint surface replacement implant and a fastening device for fixing the joint surface replacement implant in the joint gap of a facet joint of a human or animal spinal column, which fastening device comprises at least two fastening elements that protrude from the joint surface replacement implant, wherein the at least two fastening elements are each provided in the region of their free ends with at least one fixing element through hole, and wherein the facet joint implant comprises a fixing element which is fed through at least two fixing element through holes of at least two fastening elements, wherein the free ends of said fixing element are connectable to one another.

2. A facet joint implant in accordance with claim 1, wherein the fixing element is flexible.

3. A facet joint implant in accordance with claim 1, wherein the fixing element is in the form of a thread or a wire.

4. A facet joint implant in accordance with claim 1, wherein the at least two fastening elements are in the form of fastening straps, tabs, strips or loops or eyelets.

5. A facet joint implant in accordance with claim 1, wherein free ends of the at least two fastening elements are at least one of thickened and reinforced or wherein the at least two fastening elements are at least one of thickened and reinforced in the region of their free ends.

6. A facet joint implant in accordance with claim 1, wherein the fixing element through holes are at least one of thickened and reinforced.

7. A facet joint implant in accordance with claim 1, further comprising a fixing element locking arrangement for connecting the free ends of the fixing element by the application of a tensile-load.

8. A facet joint implant in accordance with claim 7, wherein the fixing element locking arrangement is in the form of a knot, a seal or a clip.

9. A facet joint implant in accordance with claim 1, further comprising four to 25 fastening elements.

10. A facet joint implant in accordance with claim 1, wherein the at least two fastening elements are at least one of a differing length, a differing breadth, and a differing thickness.

11. A facet joint implant in accordance with claim 1, wherein the at least two fastening elements are in the form of different kinds of fastening elements.

12. A facet joint implant in accordance with claim 1, further comprising at least one retaining element for digging into bone tissue.

13. A facet joint implant in accordance with claim 12, wherein the at least one retaining element is arranged or formed on at least one of the joint surface replacement implant and at least one of the fastening elements.

14. A facet joint implant in accordance with claim 12, wherein the at least one retaining element is in the form of a barb or a pointed projection.

15. A facet joint implant in accordance with claim 1, wherein at least one of the joint surface replacement implant and the fastening device is made entirely or partly of a metal or a synthetic material.

16. A facet joint implant in accordance with claim 15, wherein at least one of the joint surface replacement implant and the fastening device is made entirely or partly of a synthetic material, wherein the synthetic material comprises at least one of polyetheretherketone and polyethylene.

17. A facet joint implant in accordance with claim 15, wherein at least one of the joint surface replacement implant and the fastening device is made entirely or partly of a synthetic material, wherein the synthetic material is at least one of a thermally activatable and a thermally shrinkable synthetic material.

18. A facet joint implant in accordance with claim 15, wherein at least one of the joint surface replacement implant and the fastening device is made entirely or partly of a synthetic material, and wherein at least one of parts or regions of at least one of the joint surface replacement implant and the fastening device that are made from the synthetic material are provided with an osteo-integrative coating.

19. A facet joint implant in accordance with claim 1, wherein the at least two fastening elements are flat or slightly curved in a basic position.

20. A facet joint implant in accordance with claim 1, wherein the at least two fastening elements are at least one of flexible and resilient.

21. A facet joint implant in accordance with claim 1, wherein the at least two fastening elements and the joint surface replacement implant are connected to one another in an articulated manner.

22. A facet joint implant in accordance with claim 21, further comprising a joint for the articulated connection of one of the fastening elements to the joint surface replacement implant.

23. A facet joint implant in accordance with claim 22, wherein the joint is in the form of a hinge joint.

24. A facet joint implant in accordance with claim 1, wherein the facet joint implant is formed in a one piece manner.

25. A facet joint implant in accordance with claim 1, wherein the joint surface replacement implant and the at least two fastening elements are formed in a one piece manner.
